(19) United States
(12) Patent Application Publication
(10) Pub. No.: US 2008/0286211 A1
(43) Pub. Date: Nov. 20, 2008

(54) Using mucin glycoproteins in combination with therapeutic agents to treat epithelial lesions and disorders of impaired mucin function

(76) Inventor: Nicholas P. Barker, Southborough, MA (US)

Correspondence Address: CLARK & ELBING LLP, 101 FEDERAL STREET, BOSTON, MA, 02110, US

(21) Appl. No.: 12/150,082

(22) Filed: Apr. 24, 2008

Related U.S. Application Data
(60) Provisional application No. 60/914,692, filed on Apr. 27, 2007.

(51) Int. Cl. A61K 9/12; C07K 2/00; A61K 38/17; A61P 27/02; A61P 1/02; A61K 38/02

(52) U.S. Cl. 424/45; 514/8; 514/12

(57) ABSTRACT

The present invention features methods of treating a patient having an epithelial lesion or disorder of impaired mucin function. The present invention further features methods of treating pain associated with epithelial lesions and disorders of impaired mucin function. Epithelial lesions and disorders of impaired mucin function can be treated using a pharmaceutical composition containing mucin glycoproteins in combination with therapeutic agents, e.g., trefoil polypeptides.

### CROSS REFERENCE TO RELATED APPLICATIONS

This application claims the benefit of U.S. Provisional Application No. 60/914,692, filed Apr. 27, 2007, which is hereby incorporated by reference.

### BACKGROUND OF THE INVENTION

In general, this invention relates to the treatment of epithelial lesions and mucin disorders.

Mucin glycoproteins are gel-forming polypeptides that serve as a dense, protective barrier on cell surfaces. This mucosal barrier formed by mucin glycoproteins prevents the entrance of pathogens and large macromolecules into the cell, assists with the transport of proteins needed for the growth and repair of the epithelium, and facilitates the retention of water at mucosal surfaces.

Disorders of impaired mucin function can result, in some cases, in the formation of epithelial lesions. These lesions may provide a portal of entry for microorganisms and potentially may lead to debilitating local infections or life-threatening systemic infection. There exists a need in the art for improved methods and compositions to treat and prevent epithelial lesions and disorders of impaired mucin function.

### SUMMARY OF THE INVENTION

The invention features methods, compositions, and kits for treating epithelial lesions or disorders of impaired mucin function. The present invention is also useful for treating pain associated with epithelial lesions or disorders of impaired mucin function.

Accordingly, the invention features a method for treating a patient having an epithelial lesion. The method includes administering to a patient a pharmaceutical composition that contains a mucin glycoprotein and a therapeutic agent, wherein the mucin glycoprotein and therapeutic agent are present in the composition in amounts that are sufficient to treat an epithelial lesion or a disorder of impaired mucin function. The pharmaceutical composition containing the mucin glycoprotein and therapeutic agent, described herein, may also be used to treat pain associated with an epithelial lesion or disorder of impaired mucin function. The epithelial lesion or disorder of impaired mucin function may affect the epithelium of, e.g., the stomach (e.g., intestines or distal bowel), mouth, esophagus, skin, vagina, cervix, uterus, or eye (e.g., the cornea), or the lesion or disorder may affect the respiratory epithelium. The lesion may be the result of radiation therapy or chemotherapy. The composition may be administered to the lesion of the patient (e.g., a human) or may be administered to a site of impaired mucin function in the patient.

The invention also features a kit containing a pharmaceutical composition that includes a mucin glycoprotein and a therapeutic agent and instructions for administering the composition to a patient having an epithelial lesion or disorder of impaired mucin function.

The pharmaceutical composition may be sterile and formulated in unit dosage form. The composition may be formulated for, e.g., oral, buccal, topical, rectal, subcutaneous, vaginal, inhalation, ophthalmic, parenteral, intravenous, or intramuscular administration. The pharmaceutical composition may be formulated as, e.g., a pill, a powder, a granulate, a suspension, an emulsion, a solution, a gel, a paste, an ointment, a cream, a foam, a lotion, a plaster, a suppository, an enema, an injectable, an implant, a spray, or an aerosol. The composition may be formulated for targeted delivery or extended release.

The mucin glycoprotein of the methods, compositions, and kits described herein may include, e.g., MUC1, MUC2, MUC3A, MUC3B, MUC4, MUC5AC, MUC5B, MUC6, MUC7, MUC8, MUC9, MUC11, MUC12, MUC13, MUC15, MUC16, MUC17, MUC18, MUC19, or MUC20. The therapeutic agent of the methods, compositions, and kits described herein may be selected from, e.g., a trefoil polypeptide, a chemotherapeutic agent, an anti-inflammatory agent, an antimicrobial agent, an antiviral agent, an antifungal agent, an analgesic, an anesthetic, a sedative, a lubricant, an immunomodulatory agent, a 5-aminosalicylate derivative, or a peptide. In some instances, the therapeutic agent is not suitable for ophthalmic administration. An additional therapeutic agent may be administered to a patient concurrently or within fourteen days of administering the pharmaceutical composition.

The trefoil polypeptide described herein may be selected, e.g., from intestinal trefoil factor (ITF; SEQ ID NO.: 1), pS2 (SEQ ID NO: 2), and spasmolytic peptide (SP; SEQ ID NO: 3), or any biologically active fragments of these polypeptides. In some instances, the trefoil polypeptide is selected from the group consisting of ITF₁₋₇₂, ITF₁₅₋₇₂, ITF₁₅₋₇₃, ITF₂₁₋₆₂, ITF₂₁₋₇₂, ITF₂₁₋₇₃, ITF₂₂₋₆₂, ITF₂₂₋₇₀, ITF₂₂₋₇₂, ITF₂₂₋₇₃, ITF₂₅₋₆₂, ITF₂₅₋₇₀, ITF₂₅₋₇₂, and ITF₂₅₋₇₃, wherein the subscripts delineate the bounds of each polypeptide according to SEQ ID NO.: 1. Additionally, the trefoil polypeptide described herein can be substantially identical to SEQ ID NO.: 1, SEQ ID NO.: 2, or SEQ ID NO.: 3 over at least 10, 15, 20, 25, 30, 35, 40, 45, 50, 55, 60, 65, or 70 contiguous residues. The degree of sequence identity can be, e.g., 70%, 75%, 80%, 85%, 90%, 95%, 98%, 99%, or even 100%. Trefoil polypeptides may be administered according to any of the methods described herein as a monomer, a dimer, or in another multimeric form.

By “administering” is meant a method of giving a dosage of a pharmaceutical composition to a patient. The compositions utilized in the methods described herein can be administered by a route selected from, e.g., ocular, inhalation, parenteral, dermal, transdermal, buccal, rectal, vaginal, sublingual, perilingual, nasal, topical administration, and oral administration. Parenteral administration includes intravenous, intraperitoneal, subcutaneous, and intramuscular administration. The preferred method of administration can vary depending on various factors, e.g., the components of the composition being administered and the severity of the condition being treated.

By “an amount sufficient to treat” is meant the amount of a compound required to improve, inhibit, or ameliorate a condition of a patient, or a symptom of a disorder, in a clinically relevant manner. Any improvement in the patient is considered sufficient to achieve treatment. A sufficient amount of an active compound used to practice the methods described herein, e.g., the treatment of an epithelial lesion or the treatment of a disorder of impaired mucin function, varies depending upon the manner of administration and the age, body weight, and general health of the patient. Ultimately, the prescribers or researchers will decide the appropriate amount and dosage regimen.

By “chemotherapy” is meant the use of a chemotherapeutic agent to destroy a cancer cell, or to slow, arrest, or reverse the growth of a cancer cell.

By “chemotherapeutic agent” is meant a chemical that may be used to destroy a cancer cell, or to slow, arrest, or reverse the growth of a cancer cell. Chemotherapeutic agents include, e.g., asparaginase, bleomycin, busulfan carmustine (BCNU), chlorambucil, cladribine (2-CdA), CPT11, cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thiogaunine, topotecan, vinblastine, and vincristine.

By “disorder of impaired mucin function” is meant any disease or disorder that results in an alteration in a characteristic of mucin produced at a particular epithelial surface, e.g., the quantity, quality, type, or glycosylation of the mucin, as compared to the mucin that would be produced at that surface in a healthy individual. Disorders of impaired mucin function may include, e.g., corneal disorders (e.g., dry eye), vaginal disorders (e.g., vaginal dryness), inflammatory bowel disease, sequelae of chemotherapy or radiotherapy (e.g., reduction in saliva production), and acute or chronic airway disease. In some disorders of impaired mucin function, the mucin quantity or other characteristic may be increased or decreased, in comparison with a healthy individual, by, e.g., 5%, 10%, 20%, 30%, 40%, 50%, 60%, 70%, 80%, 90%, 95%, or 100% as measured by any standard technique.

By “epithelial lesion” is meant an area of altered epithelial tissue, e.g., a wounded, diseased, or disordered epithelial region. A lesion may be either noncancerous or cancerous. Lesions may occur on any epithelial surface, e.g., the surface of the eye, the skin, the vaginal, cervical, or uterine epithelium, the alimentary canal (including the oral cavity, esophagus, stomach, large intestine, small intestine, and colon), and the respiratory epithelium. Surfaces of internal organs, e.g., the cardiovascular system, may also manifest epithelial lesions. Epithelial lesions include, e.g., incisions or other surgical procedures, ulcers, burns, scrapes, cuts, or epithelial regions infected by bacteria, viruses, or fungi.

By a “high dosage” is meant at least 5% more (e.g., at least 10%, 20%, 50%, 100%, 200%, or even 300%) than the highest standard recommended dosage of a particular compound formulated for a given route of administration for treatment of any human disease or condition. For example, a high dosage of a therapeutic agent formulated for ocular administration may differ from a high dosage of the same agent formulated for intravenous administration.

By “immunomodulatory agent” is meant an agent that can elicit or suppress an immune response. Examples of immunomodulatory agents include, e.g., non-steroidal immunophilin-dependent immunosuppressants, e.g., cyclosporine (e.g., Restasis), and steroids, e.g., dexamethasone, rimexolone, fluorometholone, medrysone, and loteprednol etabonate.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition.

By “mucin glycoprotein” is meant a biologically active, glycosylated polypeptide that is substantially identical, e.g., at least 60%, 65%, 70%, 75%, 80%, 85%, 90%, 95%, 98%, 99%, or even 100% identical, to MUC1, MUC2, MUC3A, MUC3B, MUC4, MUC5AC, MUC5B, MUC6, MUC7, MUC8, MUC9, MUC11, MUC12, MUC13, MUC15, MUC16, MUC17, MUC18, MUC19, or MUC20 over at least 20, 40, 60, 80, 100, 200, 300, 400, or 500 contiguous residues. Carbohydrates may contribute, e.g., 50-90% of the total molecular weight of the mucin glycoprotein. A mucin glycoprotein is biologically active if it exhibits a biological activity of a naturally-occurring mucin glycoprotein, e.g., the ability to form a mucus.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “patient” is meant any animal, e.g., mammal (e.g., a human).

A patient who is being treated according to the methods described herein, e.g., a patient having an epithelial lesion or a disorder of impaired mucin function, is one who has been diagnosed by a medical practitioner as having such a condition. Diagnosis may be performed by any suitable means. A patient in whom the development of an epithelial lesion or a disorder of impaired mucin function is being prevented may or may not have received such a diagnosis. One skilled in the art will understand that patients described herein may have been subjected to standard tests or may have been identified, without examination, as one at high risk due to the presence of one or more risk factors, such as age or a family history of epithelial lesions or disorders of impaired mucin function.

By “pharmaceutical composition” is meant any composition that contains at least one therapeutically or biologically active agent and is suitable for administration to a patient. For the purposes of this invention, pharmaceutical compositions suitable for delivering a therapeutic can include, e.g., eye drops, tablets, gelcaps, capsules, pills, powders, granulates, suspensions, emulsions, solutions, gels, hydrogels, oral gels, pastes, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

The terms “polypeptide” and “peptide” are used interchangeably and refer to any chain of more than two natural or unnatural amino acids, regardless of post-translational modification (e.g., glycosylation or phosphorylation), constituting all or part of a naturally-occurring or non-naturally occurring polypeptide or peptide, as is described herein.

As used herein, a natural amino acid is a natural α-amino acid having the L-configuration, such as those normally occurring in natural proteins. Unnatural amino acid refers to an amino acid, which normally does not occur in proteins, e.g., an epimer of a natural α-amino acid having the L configuration, that is to say an amino acid having the unnatural D-configuration; or a (D,L)-isomeric mixture thereof; or a homologue of such an amino acid, for example, a β-amino acid, an α,α-disubstituted amino acid, or an α-amino acid wherein the amino acid side chain has been shortened by one or two methylene groups or lengthened to up to 10 carbon atoms, such as an α-amino alkanoic acid with 5 up to and including 10 carbon atoms in a linear chain, an unsubstituted or substituted aromatic (α-aryl or α-aryl lower alkyl), for example, a substituted phenylalanine or phenylglycine.

By “radiation therapy” is meant the use of radiation, e.g., directed gamma rays or X-rays, to induce sufficient damage to a cell so as to limit its ability to function normally or to destroy the cell altogether. Methods of determining the dosage and duration of radiation therapy are well-known in the art.

By “substantially identical” is meant a polypeptide or nucleic acid exhibiting at least 60%, 65%, 70%, 75%, 80%, 85%, 90%, 95%, 98%, 99%, or even 100% identity to a reference amino acid or nucleic acid sequence over at least 20, 25, 30, 35, 40, 45, 50, 55, 60, 65, or 70 contiguous residues or bases. For example, the trefoil polypeptide fragment amino acid sequences GLSANQCAVPAKDRVDCGYP and GLSPSQCMAPTNVRVDCNYP share 60% identity over 20 residues.

Sequence identity is typically measured using a sequence analysis program (e.g., BLAST 2; Tatusova et al., FEMS Microbiol Lett. 174:247-250, 1999) with the default parameters specified therein. Conservative substitutions typically include substitutions within the following groups: glycine, alanine, valine, isoleucine, leucine; aspartic acid, glutamic acid, asparagine, glutamine; serine, threonine; lysine, arginine; and phenylalanine and tyrosine.

By “therapeutic agent” is meant any agent that produces a preventative, healing, curative, stabilizing, or ameliorative effect.

By “treating” is meant administering a pharmaceutical composition for prophylactic and/or therapeutic purposes. Prophylactic treatment may be administered, for example, to a subject who is not yet ill, but who is susceptible to, or otherwise at risk of, a particular disorder, e.g., an epithelial lesion or a disorder of impaired mucin function. Therapeutic treatment may be administered, for example, to a subject already suffering from a disorder in order to improve or stabilize the subject's condition. Thus, in the claims and embodiments described herein, treating is the administration to a subject either for therapeutic or prophylactic purposes. In some instances, as compared with an equivalent untreated control, treatment may ameliorate a disorder or a symptom thereof, such as pain, by, e.g., 5%, 10%, 20%, 30%, 40%, 50%, 60%, 70%, 80%, 90%, 95%, or 100% as measured by any standard technique. In some instances, treating can result in the inhibition of the formation of lesions, the healing of lesions already formed, and the amelioration of a disorder of impaired mucin function. For example, mucin glycoproteins in combination with, e.g., trefoil polypeptides (e.g., ITF₁₅₇₃ and ITF₂₁₋₇₃), that promote healing of epithelial lesions or amelioration of disorders of impaired mucin function, are useful in the methods described herein.

By “trefoil polypeptide” is meant a biologically active polypeptide or any biologically active fragment of a polypeptide that is substantially identical, e.g., at least 60%, 65%, 70%, 75%, 80%, 85%, 90%, 95%, 98%, 99%, or even 100% identical, to intestinal trefoil factor (ITF; SEQ ID NO.: 1), pS2 (SEQ ID NO: 2), or spasmolytic peptide (SP; SEQ ID NO: 3) over at least 20, 25, 30, 35, 40, 45, 50, 55, 60, 65, or 70 contiguous residues. A trefoil polypeptide is biologically active if it exhibits a biological activity of a naturally-occurring trefoil polypeptide, e.g., the ability to alter gastrointestinal motility in a mammal or the ability to enhance corneal epithelial wound healing. Exemplary trefoil polypeptides useful in the invention are ITF₅₋₇₃ and ITF₂₁₋₇₃. Other trefoil polypeptides useful in the invention include ITF₁₋₇₂, ITF₁₅₋₇₂, ITF₂₂₋₆₂, ITF₂₁₋₇₂, ITF₂₂₋₆₂, ITF₂₂₋₇₀, ITF₂₂₋₇₂, ITF₂₂₋₇₃, ITF₂₅₋₆₂, ITF₂₅₋₇₀, ITF₂₅₋₇₂, and ITF₂₅₋₇₃, wherein the subscripts delineate the bounds of each polypeptide according to SEQ ID NO.: 1. Trefoil polypeptides may occur as monomers, dimers, or other multimeric forms.

Other features and advantages of the invention will be apparent from the detailed description and from the claims.

### BRIEF DESCRIPTION OF THE DRAWINGS

FIG. 1 shows the amino acid sequence (Accession No. BAA95531; SEQ ID NO.: 1) of human intestinal trefoil factor.

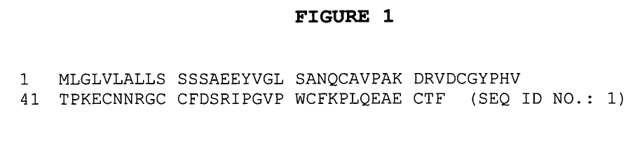

FIG. 2 shows the amino acid sequence (Accession No. NP_{—}003216; SEQ ID NO.: 2) of human pS2 protein.

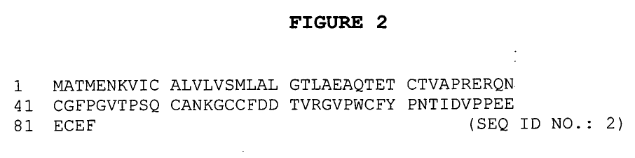

FIG. 3 shows the amino acid sequence (Accession No. 1909187A; SEQ ID NO.: 3) of human spasmolytic polypeptide (SP).

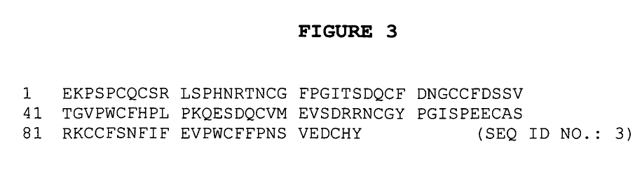

### DETAILED DESCRIPTION OF THE INVENTION

The present invention features methods, compositions, and kits for treating a patient having an epithelial lesion or disorder of impaired mucin function. Epithelial lesions and disorders of impaired mucin function can be treated, e.g., using a pharmaceutical composition containing mucin glycoproteins in combination with therapeutic agents, e.g., trefoil polypeptides.

### Diagnosis and Treatment of Epithelial Lesions and Disorders of Impaired Mucin Function

Epithelial lesions and impaired mucin function treated by the methods and compositions described herein may be caused by or associated with, e.g., a disorder, a surgical procedure, or an injury. Epithelial lesions and impaired mucin function may affect, e.g., the stomach (e.g., intestinal or distal bowel), oral, esophageal, skin, vaginal, cervical, uterine, eye, or respiratory epithelium.

Stomach Lesions and Disorders

The methods and compositions described herein may be used to treat epithelial lesions of the stomach, intestines, and distal bowel caused by physical trauma, surgical intervention (e.g., biopsy, resection, or hemorrhoidectomy), or antineoplastic therapy (e.g., chemotherapy or radiation therapy). Additionally, lesions of the stomach, intestines, or distal bowel that result from microbial (e.g., bacterial, viral, or fungal) infection may also be treated. Many disorders of the stomach may result in epithelial lesions or impaired mucin function and include, e.g., inflammatory bowel disease, Crohn's disease, ulcerative colitis, gastric cancer, colorectal cancer, Hirschsprung's disease, enterocolitis, proctitis, and enteritis.

Oral Lesions and Disorders

Oral lesions may be the result of, e.g., gingivitis, tooth extraction, bite injuries, oral biopsies, oral surgery, thermal burns (e.g., resulting from the ingestion of overheated food or drink), or chemical bums (e.g., resulting from the ingestion of acidic food or associated with industrial or occupational exposures). Disorders resulting in epithelial lesions or impaired mucin function may include, e.g., mucositis, aphthous stomatitis, Behcet's disease, and microbial (e.g., bacterial, viral, or fungal) infections.

Skin Lesions and Disorders

Epithelial lesions may occur on any part of the human skin, including, e.g., the scalp, groin and uro-genital area, face, trunk, arms, hands, legs, soles of the feet, or between the toes. Lesions of the skin can be induced by physical trauma (e.g., cuts, abrasions, or surgical intervention), chemical and thermal burns (e.g., sunburn), vascular compromise (e.g., resulting from diabetes), infective or inflammatory processes (e.g., eczema, psoriasis, contact dermatitis, herpetic lesion, or acne), microbial infection (e.g., viral, bacterial, and fungal), or antineoplastic therapy (e.g., chemotherapy or radiation therapy).

Vaginal, Cervical, and Uterine Lesions and Disorders

Vaginal, cervical, and uterine epithelial lesions may be induced by physical trauma, including those resulting from sexual intercourse, contact with a foreign object, surgical intervention (e.g., episiotomy, hysterectomy, or biopsy), or vaginal childbirth. Epithelial lesions may be caused by an infection (e.g., bacterial, viral, or fungal), systemic or local chemotherapy, or pelvic radiation therapy. Disorders such as, e.g., vaginal dryness, atrophic vaginal mucosa, pelvic inflammatory disease (PID), bacterial infections by, e.g., N. gonorrhea, T. pallidum, Gardnerella spp., and Chlamydia spp., viral infections by, e.g., herpes simplex virus and papilloma virus, or fungal infections by, e.g., Candida albicans, may result in epithelial lesions or impaired mucin function.

Respiratory Epithelium Lesions and Disorders

Lesions of the respiratory epithelium that may be treated according to the methods and compositions described herein may be caused by physical trauma (e.g., surgical intervention or intubation), chemical trauma (e.g., smoking or exposure to a volatile solvent), thermal trauma, vascular compromise (e.g., resulting from congestive heart failure or chronic obstructive pulmonary disease), infective or inflammatory processes, antineoplastic therapy (e.g., radiation therapy or chemotherapy), diseases processes (e.g., cystic fibrosis or asthma), or exposure to high concentrations of oxygen (e.g., hyperbaric oxygen therapies) for extended periods of time. Lesions of the epithelium may result from allergic reactions. Respiratory disorders such as, e.g., cystic fibrosis, primary ciliary dyskinesia, chronic obstructive pulmonary disease, asthma, or acute or chronic airway disease may result in epithelial lesions or impaired mucin function.

Ophthalmic Lesions and Disorders

An ophthalmic procedure, e.g., radial keratotomy, astigmatic or arcuate keratotomy, laser thermokeratoplasty, conductive keratoplasty, photorefractive keratectomy (PRK), phototherapeutic keratectomy, photoastigmatic refractive keratectomy, hyperopic photorefractive keratectomy, presbyopic photorefractive keratectomy, laser-assisted in situ keratomileusis (LASIK), hyperopic-LASIK, epi-LASIK, intra-LASIK, wavefront-LASIK, laser-assisted subepithelial keratomileusis, phakic intraocular lens implant, intraocular contact lens implant, refractive lens exchange, cataract extraction and intraocular lens implant, intracorneal ring implant, scleral expansion, or limbal relaxing incision, may result in an epithelial lesion.

An ophthalmic disorder may affect any part of the eye, e.g., the cornea, the sclera, the retina, the conjunctiva, the ciliary body, the posterior chamber, or the anterior chamber. In some instances, the ophthalmic disorder affects the cornea, e.g., the corneal epithelium, or the conjunctiva. Ophthalmic disorders that may be associated with epithelial lesions or impaired mucin function include, e.g., superficial punctate keratitis, corneal ulcer, herpes simplex keratoconjunctivitis, ophthalmic herpes zoster, phlyctenular keratoconjunctivitis, keratoconus, conjunctiva, keratoconjunctivitis sicca (dry eyes), ocular inflammation, corneal ulcers, and cicatricial pemphigoid. Ophthalmic disorders can be caused by viruses (e.g., adenoviruses or herpes simplex virus), blepharitis, keratitis sicca, trachoma, corneal foreign bodies, ultraviolet light exposure (e.g., welding arcs or sunlamps), contact lens overwear, systemic drugs (e.g., adenine arabinoside), topical drugs, bacteria, protozoa, fungi, or by a hypersensensitive reaction to a known or unknown antigen. Physical eye trauma can also result in an ophthalmic disorder. Physical trauma to the eye includes an abrasion to the cornea (e.g., caused by a foreign body), perforation of the cornea (e.g., caused by a blunt injury that disrupts the continuity of the cornea), chemical burns to the cornea (e.g., exposure to NaOH), or through surgical procedures (e.g., corneal transplants and intraocular injections). The ophthalmic disorder generally results in damage and disruption of eye function or structure. For example, the disorder may cause the corneal epithelium to tear, may cause necrosis of the cornea, may cause corneal ulcers, or may damage the conjunctiva.

Diagnosis of Epithelial Lesions and Disorders of Impaired Mucin Function

Epithelial lesions or disorders of impaired mucin function may require treatment, e.g., prophylactic or therapeutic treatment. Any of the methods known to those skilled in the art may be used to diagnosis the epithelial lesion or disorder of impaired mucin function and to determine an appropriate course of therapy. Epithelial lesions and disorders of impaired mucin function may be treated with mucin glycoproteins in combination with therapeutic agents, e.g., trefoil polypeptides.

### Therapy

Therapy according to the methods described herein may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the severity of the patient's epithelial lesion or disorder of impaired mucin function, and how the patient responds to the treatment.

In order to measure the efficacy of any of the methods or compositions described herein, a pain measurement index may be used. Indices that may be useful in the methods and compositions described herein include a visual analog scale (VAS) or a Likert scale, each of which is well-known in the art. Such indices may be used to measure pain, tenderness, light sensitivity, itchiness, burning sensations, eye-pain sensations, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales and their use are described, e.g., in U.S. Pat. No. 6,623,040 and U.S. Pat. No. 6,766,319.

### Formulation and Administration of Pharmaceutical Compositions

The pharmaceutical compositions described herein are prepared in a manner known to those skilled in the art, for example, by means of conventional dissolving, lyophilizing, mixing, granulating, or confectioning processes. Methods well-known in the art for making formulations are found, for example, in Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

Administration of compositions described herein may be by any suitable means that results in a compound concentration that is effective for treating or inhibiting (e.g., by delaying) the development of an epithelial lesion or impaired mucin function. Mucin glycoprotein and a therapeutic agent are admixed with a suitable carrier substance, e.g., a pharmaceutically acceptable excipient that preserves the therapeutic properties of the compounds with which it is administered. One exemplary pharmaceutically acceptable excipient is physiological saline. The suitable carrier substance is generally present in an amount of 1-95% by weight of the total weight of the composition.

The compositions described herein may include a purified mucin, e.g., MUC1 or MUC2. Mucin solutions may be prepared by dissolving the mucin glycoprotein in any suitable diluent, e.g., sterile water or saline, buffered to a pH between 6.5 and 7.4. The concentration of the mucin glycoprotein in the solution may be, e.g., 0.01, 0.1, 1, 5, 10, 20, 30, 40, 50, 60, 70, 80, 90, 95, 98, or 99% of the administered composition. The therapeutic agent, e.g., trefoil polypeptide, may be dissolved in the same diluent as the mucin glycoprotein or a different diluent. The therapeutic agent used in the methods and compositions described herein may be provided in therapeutically effective amounts. For example, the concentration of trefoil peptide in the pharmaceutical composition may be, e.g., 0.05, 0.1, 0.5, 1, 5, 10, 15, 20, 25, 30, 35, 40, 45, 50, 60, 70, 80, 90, or 100 mg/ml. The composition may contain, e.g., 0.1, 0.5, 1, 2, 3, 4, 5, 6, 7, 8, 9, 10, 20, 50, or 100 mM of a divalent cation, e.g., CaCl₂.

The pharmaceutical compositions described herein may contain a buffering agent. Any pharmaceutically acceptable buffering agent may be used in the composition, including, e.g., phosphate, borate, citrate, acetate, or carbonate. Preferably, the buffering agent will produce a pH between 6.5 and 7.4. Suitable acids (e.g., hydrochloric acid) and bases (e.g., sodium hydroxide) may be used to adjust the pH of the pharmaceutical composition. Tonicity agents may be added to adjust the tonicity of the pharmaceutical composition with respect to the tonicity at the site of administration. Exemplary tonicity agents include, e.g., sodium chloride, potassium chloride, dextrose, mannitol, and sorbitol. Humectants, or water-binding compounds, may be added to the compositions described herein to aid in the retention of moisture. Exemplary humectants include, e.g., glycerin, propylene glycol, and polyethylene glycol. Humectants may also be added to, e.g., surfaces to which the pharmaceutical composition is applied to keep the surface hydrated and moist. The pharmaceutical composition may include preservatives in an amount sufficient to prevent microbial growth in the composition when stored. Preservatives may include, e.g., benzylkonium chloride, chlorohexidine gluconate, polyhexamethylene biguanide, and ascorbic acid.

The composition may be provided in a dosage form that is suitable for, e.g., ocular, inhalation, parenteral, dermal, transdermal, buccal, rectal, vaginal, sublingual, perilingual, nasal, topical administration, and oral administration. Parenteral administration includes intravenous, intraperitoneal, subcutaneous, and intramuscular administration. Thus, the composition may be in form of, e.g., eye drops, tablets, gelcaps, capsules, pills, powders, granulates, suspensions, emulsions, solutions, gels, hydrogels, oral gels, pastes, ointments, creams, plasters, drenches, delivery devices, suppositories, enemas, injectables, implants, sprays, or aerosols. In some instances, the composition is suitable for opthalmic use, e.g., in the form of an eye drop or ointment. Alternatively, in some instances, the composition is not suitable for ophthalmic use. The composition may be sterile. The composition may be in the form of a liquid, semi-solid, or solid. The pharmaceutical composition may be, e.g., a swell-controlled, slow-release gastric, intestinal, or colonic preparation.

Pharmaceutical compositions according to the invention may be formulated to release the active compound immediately upon administration (e.g., targeted delivery), or at any predetermined time period after administration, using controlled or extended release formulations. Administration of compounds in controlled or extended release formulations is useful where the compound, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the therapeutic compound. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

### Dosages

The pharmaceutical composition described herein may be administered once, twice, three times, four times, or five times each day, or in other quantities and frequencies. Alternatively, the pharmaceutical composition may be administered once per week, twice per week, three times per week, four times per week, five times per week, or six times per week. Therapy with the composition described herein can continue until the epithelial lesion has healed or the disorder of impaired mucin function has been ameliorated. The duration of therapy can be, e.g., one week to one month; alternatively, the pharmaceutical composition can be administered for a shorter or a longer duration. Continuous daily dosing with compounds used in the methods described herein may not be required. A therapeutic regimen may require cycles, during which time a composition is not administered, or therapy may be provided on an as-needed basis.

Appropriate dosages of compounds used in the methods described herein depend on several factors, including the administration method, the severity of the epithelial lesion or disorder of mucin function, and the age, weight, and health of the patient to be treated. Additionally, pharmacogenomic information (e.g., the effect of genotype on the pharmacokinetic, pharmacodynamic, or efficacy profile of a therapeutic) about a particular patient may affect the dosage used.

### Mucin Glycoproteins

Mucin glycoproteins are large, gel-forming polypeptides linked to carbohydrates. Mucins may play a protective role by keeping pathogens away from the surface epithelia of cells and forming a dense barrier to prevent access of pathogens and large macromolecules to the epithelial surface (see, e.g., Gipson and Argueso, Int. Rev. Cytology, 231:1-49, 2003, hereby incorporated by reference).

Mucin glycoproteins useful in the methods, compositions, and kits described herein can include either secreted mucins or membrane-anchored mucins. Exemplary mucins are MUC1, MUC2, MUC3A, MUC3B, MUC4, MUC5AC, MUC5B, MUC6, MUC7, MUC8, MUC9, MUC11, MUC12, MUC13, MUC15, MUC16, MUC17, MUC18, MUC19, and MUC20. The secretory mucins include, e.g., MUC2, MUC5AC, MUC5B, MUC6, and MUC7, and are gel-forming and rich in cysteine (see, e.g., Rose and Voynow, Physiol. Rev. 86:245-78, 2006, hereby incorporated by reference). Membrane-associated mucins also form gels and are the major constituent of the glycocalyx on the surface of epithelial cells. In both secreted and membrane-associated mucins, carbohydrates generally contribute 50-90% of the total weight of the mucin glycoprotein. In some instances, the mucin glycoproteins described herein may constitute a viable biological matrix for binding specific classes of therapeutic agents, e.g., peptides such as trefoil polypeptides, α-defensins, β-defensins, cathelicidins, cathepsins, and other therapeutic peptides (see, e.g., Moal et al., Clin. Microbiol. Rev. 19:315-37, 2006, hereby incorporated by reference).

Mucin glycoproteins useful in the methods, compositions, and kits described herein can include, e.g., fragments, allelic variations, homologs from other species, e.g., rat or mouse homologs, natural mutants, induced mutants, proteins encoded by DNA that hybridizes under high or low stringency conditions to mucin glycoprotein-encoding nucleic acids retrieved from naturally occurring material, and polypeptides retrieved by antisera to mucin glycoprotein. Mucin glycoprotein can also include chimeric polypeptides that include a mucin glycoprotein as defined herein, fused to another domain, e.g., with a separate activity or characteristic.

Mucin glycoproteins useful in the methods, compositions, and kits described herein can include modifications, e.g., in vivo or in vitro chemical derivatization of polypeptides, e.g., acetylation or carboxylation. Also included are modifications of glycosylation, e.g., those made by modifying the glycosylation patterns of a polypeptide during its synthesis and processing or in further processing steps, e.g., by exposing the polypeptide to enzymes that affect glycosylation derived from cells that normally provide such processing, e.g., mammalian glycosylation enzymes. Also encompassed are versions of the same primary amino acid sequence that have phosphorylated amino acid residues, e.g., phosphotyrosine, phosphoserine, or phosphothreonine. Mucin glycoproteins can differ from naturally-occurring mucin glycoproteins by alterations of their primary sequence. These include genetic variants, both natural and induced. Induced mutants may be derived by various techniques, including random mutagenesis of the encoding nucleic acids using irradiation or exposure to ethanemethylsulfate (EMS), or may incorporate changes produced by site-specific mutagenesis or other techniques of molecular biology. See, e.g., Sambrook and Russell, Molecular Cloning: A Laboratory Manual (3rd edition), Cold Spring Harbor Laboratory Press (2001), hereby incorporated by reference. Also included are mucin glycoproteins that include residues other than naturally-occurring L-amino acids, e.g., D-amino acids or non-naturally occurring or synthetic amino acids, e.g., β or γ amino acids. Mucin glycoproteins useful in the methods, compositions, and kits described herein are described, e.g., in Gum et al., J. Biol. Chem. 269:2440-6, 1994; Tomasetto et al., Gastroenterology, 118:70-80, 2000; Newton et al., Gut 46:312-20, 2000; Ruchaud-Sparagano et al., Cell. Mol. Life. Sci. 61:1946-54, 2004; Thim et al., Eur. J. Clin. Invest., 32:519-27, 2002; Moal et al., Clin. Microbiol. Rev. 19:315-37, 2006; Davies and Carlstedt, Methods Mol. Biol. in Glycoprotein Methods and Protocols, 125:3-13, 2000; and Carraway, Methods Mol. Biol. in Glycoprotein Methods and Protocols, 125:15-26, 2000, hereby incorporated by reference.

In addition to serving as a protective barrier, the ectodomains of membrane-associated and secreted mucins have hydrophilic properties that may facilitate water retention to keep the mucosal surfaces hydrated. The hydrating gels formed by the mucin glycoproteins create a fluid-filled film over the surface epithelia to facilitate the movement of proteins needed for growth and repair of the epithelium. MUC7, for example, may be involved in the movement of numerous bactericidal proteins from the lacrimal gland acini through the lacrimal duct to the tear film (see, e.g., Paulsen et al., Int. Rev. Cytology, 249:229-79, 2006, hereby incorporated by reference).

Production of Mucin Glycoproteins

Mucin glycoproteins are naturally produced and secreted in vivo by goblet cells, secretory cells, mucus cells, and polarized mucosal epithelia, e.g., in the respiratory, gastrointestinal, endocervical, genitourinary, and ocular tracts. The expression of mucin genes is highly regulated and their distribution is tissue-specific. For example, the mucus cells of the stomach epithelium express MUC5AC, while mucus cells in the neck express MUC6. Salivary gland mucus cells and submucosal glands of the bronchus express MUC5B. Expression of mucin glycoproteins may also be regulated under certain pathological states such as, e.g., acute and chronic airway disease, inflammatory bowel disease, or corneal disorders (see, e.g., Argueso et al., Invest. Opthalmol. Vis. Sci., 43:1004-11, 2002, hereby incorporated by reference).

Mucin glycoproteins useful in the methods, compositions, and kits described herein may be purified from natural sources (e.g., porcine stomach or bovine submaxillary glands). Partially purified mucin glycoproteins are available commercially from, e.g., Sigma-Aldrich (Catalog Nos. M1778, M2378, M3895, M4503; St. Louis, Mo., USA). Mucin glycoproteins may also be produced in recombinant or non-recombinant cells lines. The overexpression of recombinant mucins is described in, e.g., Backstrom et al., Biochem. J. 376:677-86, 2003; Batra et al., J. Cell. Sci. 100:841-9, 1991; Dabbagh et al., J. Immunol. 162:6233-7, 1999; Kim et al., Mol. Pharmacol. 62:1112-8, 2002; and Link et al., J. Biotechnology 110:51-62, 2004, hereby incorporated by reference. Mucin glycoproteins may be extracted and isolated from recombinant and non-recombinant cell lines, as described in, e.g., Davies and Carlstedt, Methods Mol. Biol. in Glycoprotein Methods and Protocols, 125:3-13, 2000; Carraway, Methods Mol. Biol. in Glycoprotein Methods and Protocols, 125:15-26, 2000; and Bhavanandan et al., Glycoconjugate J., 15:37-49, 1998, hereby incorporated by reference. Characterization of isolated mucin glycoproteins may be accomplished using, e.g., solution assays, gel assays (e.g., SDS-PAGE), membrane-bound methods, antibodies, enzyme-linked immuno-sorbent assays (ELISA), or liquid-chromatography electron-spray ionization mass spectrometry (LCMS).

### Therapeutic Agents

As described herein, treatment of the epithelial lesion or impaired mucin function with the methods, compositions, and kits described herein may include treatment with a mucin glycoprotein and a therapeutic agent, e.g., a trefoil polypeptide, a chemotherapeutic agent, an anti-inflammatory agent, an antimicrobial agent, an antiviral agent, an antifungal agent, an analgesic, an anesthetic, a sedative, a lubricant, an immunomodulatory agent, a 5-aminosalicylate derivative, or a peptide.

Trefoil Polypeptides

Trefoil polypeptides are described extensively in the literature (see, e.g., Sands et al., Ann. Rev. Physiol. 58: 253-273, 1996, hereby incorporated by reference). Naturally-occurring trefoil polypeptides are expressed, e.g., in the gastrointestinal tract and have a three-loop structure formed by intrachain disulfide bonds between conserved cysteine residues. Fragments that retain the trefoil structure (i.e., the three loop structure) or that lie within regions of the protein that are highly conserved are particularly useful in the invention. Thus, such fragments can, e.g., encompass portions of ITF from about the first cysteine residue involved in a disulfide bond of the three loop structure to about the last cysteine residue involved in a disulfide bond of the three loop structure.

Trefoil polypeptides useful in the methods, compositions, and kits described herein can include, e.g., fragments, allelic variations, homologs from other species, e.g., rat or mouse homologs, natural mutants, induced mutants, proteins encoded by DNA that hybridizes under high or low stringency conditions to ITF-, pS2-, or SP-encoding nucleic acids retrieved from naturally occurring material, and polypeptides retrieved by antisera to or SP. Trefoil polypeptides can also include chimeric polypeptides that include a trefoil polypeptide domain as defined herein, fused to another domain, e.g., with a separate activity or characteristic.

Trefoil polypeptides useful in the methods, compositions, and kits described herein can include modifications, e.g., in vivo or in vitro chemical derivatization of polypeptides, e.g., acetylation or carboxylation. Also included are modifications of glycosylation, e.g., those made by modifying the glycosylation patterns of a polypeptide during its synthesis and processing or in further processing steps, e.g., by exposing the polypeptide to enzymes that affect glycosylation derived from cells that normally provide such processing, e.g., mammalian glycosylation enzymes. Also encompassed are versions of the same primary amino acid sequence that have phosphorylated amino acid residues, e.g., phosphotyrosine, phosphoserine, or phosphothreonine. Trefoil polypeptides can differ from naturally-occurring ITF, pS2, or SP by alterations of their primary sequence. These include genetic variants, both natural and induced. Induced mutants may be derived by various techniques, including random mutagenesis of the encoding nucleic acids using irradiation or exposure to ethanemethylsulfate (EMS), or may incorporate changes produced by site-specific mutagenesis or other techniques of molecular biology. See, e.g., Sambrook and Russell, Molecular Cloning: A Laboratory Manual (3^{rd} edition), Cold Spring Harbor Laboratory Press (2001), hereby incorporated by reference. Also included are trefoil polypeptides that include residues other than naturally-occurring L-amino acids, e.g., D-amino acids or non-naturally occurring or synthetic amino acids, e.g., 0 or γ amino acids. Trefoil polypeptides useful in the methods, compositions, and kits described herein are described, e.g., in U.S. Pat. Nos. 6,063,755 and 6,221,840; U.S. Publication Nos. 2003-0134797, 2003-0181383, 2003-0181384, 2003-0185838, 2003-0185839, 2003-0186880, 2003-0186882, 2003-0186886, 2003-0225250, and 2004-0171544; and U.S. application Ser. Nos. 10/362,310 and 10/457,157, each of which is hereby incorporated by reference.

Trefoil polypeptides or fragments thereof can be produced by any method known in the art for expression of recombinant proteins. In general, methods for producing trefoil polypeptides are well-known in the art; see, e.g., U.S. Publication No. 2004-0171544. For example, nucleic acids that encode the desired polypeptide may be introduced into various cell types or cell-free systems for expression thereby allowing small-, large-, and commercial-scale production, purification, and patient therapy.

Chemotherapeutic Agents

Any suitable chemotherapeutic agent may be administered. Chemotherapeutic agents suitable for the composition described herein include, e.g., asparaginase, bleomycin, busulfan carmustine (BCNU), chlorambucil, cladribine (2-CdA), CPT11, cyclophosphamide, cytarabine (Ara-C), dacarbazine, daunorubicin, dexamethasone, doxorubicin (adriamycin), etoposide, fludarabine, 5-fluorouracil (5FU), hydroxyurea, idarubicin, ifosfamide, interferon-α (native or recombinant), levamisole, lomustine (CCNU), mechlorethamine (nitrogen mustard), melphalan, mercaptopurine, methotrexate, mitomycin, mitoxantrone, paclitaxel, pentostatin, prednisone, procarbazine, tamoxifen, taxol-related compounds, 6-thioguanine, topotecan, vinblastine, and vincristine. Exemplary chemotherapeutic agents are listed in, e.g., U.S. Pat. Nos. 6,864,275 and 6,984,654.

Anti-Inflammatory Agents

Any suitable anti-inflammatory agent can be administered. Suitable anti-inflammatory agents include, e.g., non-steroidal anti-inflammatory drugs (e.g., ibuprofen or tacrolimus), cyclooxygenase-2-specific inhibitors such as rofecoxib (Vioxx®) and celecoxib (Celebrex®), topical glucocorticoid agents, and specific cytokines directed at T lymphocyte function. Additional suitable anti-inflammatory agents include flubiprofen, diclofenac, and ketarolac. Anti-inflammatory concentrations known to be effective may be used. For example, ibuprofen may be present in the composition at concentrations sufficient to deliver between 25-800 mg per day to the lesion. Exemplary anti-inflammatory agents are listed in, e.g., U.S. Pat. Nos. 7,112,578 and 7,199,119.

Antimicrobial Agents

Any of the many known antimicrobial agents can be used in the compositions described herein at concentrations generally used for these agents. Antimicrobial agents include antibacterials, antifungals, and antivirals.

Examples of antibacterial agents (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, minocycline, and tetracycline), aminoglycosides (e.g., amikacin, gentamycin, kanamycin, neomycin, streptomycin, and tobramycin), macrolides (e.g., azithromycin, clarithromycin, and erythromycin), fluoroquinolones (e.g., ciprofloxacin, lomefloxacin, moxifloxacin, and norfloxacin), and other antibiotics including chloramphenicol, clindamycin, cycloserine, isoniazid, rifampin, and vancomycin. Exemplary antimicrobial agents are listed in, e.g., U.S. Pat. Nos. 6,830,745 and 7,056,917.

Antiviral agents are substances capable of destroying or suppressing the replication of viruses. Examples of antiviral agents include 1-β-D-ribofuranosyl-1,2,4-triazole-3 carboxamide (ribavirin), 9-2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir. Exemplary antiviral agents are listed in, e.g., U.S. Pat. Nos. 6,093,550 and 6,894,033.

Antifungal agents include both fungicidal and fungistatic agents, e.g., amphotericin B, butylparaben, clindamycin, econaxole, fluconazole, flucytosine, griseofulvin, nystatin, and ketoconazole. Exemplary antifungal agents are listed in, e.g., U.S. Pat. Nos. 5,627,153 and 7,125,842.

Analgesics and Anesthetics

Any of the commonly used topical analgesics and anesthetics can be used as therapeutic agents in the invention. Examples of useful anesthetics include procaine, lidocaine, tetracaine, dibucaine, benzocaine, p-buthylaminobenzoic acid 2-(diethylamino) ethyl ester HCl, mepivacaine, piperocaine, and dyclonine. Exemplary anesthetics are listed in, e.g., U.S. Pat. Nos. 6,562,363 and 6,569,839.

Analgesics include opioids such as, e.g., morphine, codeine, hydrocodone, and oxycodone. Any of these analgesics may also be co-formulated with other compounds having analgesic or anti-inflammatory properties, such as acetaminophen, aspirin, codeine, naproxen, and ibuprofen. Exemplary analgesics are listed in, e.g., U.S. Pat. Nos. 6,869,974 and 7,202,259.

Sedatives

Sedatives are agents that suppress the central nervous system. Any of the commonly used sedatives can be used in the invention. Examples of useful sedatives include alprazolam, clonazepam, diazepam, flunitrazepam, lorazepam, nitrazepam, oxazepam, and triazolam. Exemplary sedatives are listed in, e.g., U.S. Pat. No. 5,589,475.

Lubricants

Lubricants can be used in, e.g., eye drops, to treat epithelial lesions or impaired mucin function. Examples of useful lubricants include sodium hyaluronate, proteoglycans, chondroitin sulfate, cellulose derivatives, hydroxypropylmethyl cellulose, carboxymethyl cellulose, methyl cellulose, hydroxyethyl cellulose, collagen, viscosifiers, polyvinyl alcohol, polyvinylpyrrolidone, and carboxyvinyl polymers. Exemplary lubricants are listed in, e.g., U.S. Pat. No. 7,037,469.

Immunomodulatory Agents

Immunomodulatory agents are agents that can elicit or suppress an immune response. Any of the commonly used immunomodulatory agents can be used to treat epithelial lesions or impaired mucin function. Examples of useful immunomodulatory agents include, e.g., non-steroidal immunophilin-dependent immunosuppressants, e.g., ascomycin, cyclosporine (e.g., Restasis), everolimus, pimecrolimus, rapamycin, and tacrolimus. Also included are steroids, e.g., beclomethasone, budesonide, dexamethasone, fluorometholone, fluticasone, hydrocortisone, loteprednol etabonate, medrysone, rimexolone, and triamcinolone. Exemplary steroids are listed in, e.g., U.S. Pat. Nos. 5,837,698 and 6,909,007.

5-Aminosalicylate Derivatives

5-Aminosalicylate (5-ASA) derivatives, e.g., sulfasalazine, mesalamine, olsalazine, and balsalazide, may be administered as therapeutic agents. Exemplary 5-ASA derivatives are listed in, e.g., U.S. Pat. Nos. 6,326,364 and 7,119,119.

Peptides

Peptides useful may be administered to treat epithelial lesions and impaired mucin function. Examples of useful peptides include, e.g., α-defensins (e.g., HD-1, HD-2, HD-3, HD-4, HD-5, and HD-6), β-defensins (e.g., hBD-1, hBD-2, hBD-3, hBD-4, hBD-5, and hBD-6), cathelicidins (e.g., LL-37), cathepsins, or any other therapeutic peptide. Exemplary peptides are listed in, e.g., Moal et al., Clin. Microbiol. Rev. 19:315-37, 2006, hereby incorporated by reference.

### Additional Therapeutic Regimens

If desired, the patient may also receive additional therapeutic regimens. For example, an additional therapeutic agent may be administered with the compositions described herein at concentrations known to be effective for such therapeutic agents. Particularly useful agents include, e.g., chemotherapeutic agents, anti-inflammatory agents, antimicrobial agents, antiviral agents, antifungal agents, analgesics, anesthetics, sedatives, lubricants, immunomodulatory agents, 5-aminosalicylate derivatives, and peptides, described herein.

Additional therapeutic agents may be delivered separately or may be admixed into a single formulation together with the pharmaceutical composition. When agents are present in different pharmaceutical compositions, different routes of administration may be employed. Routes of administration include, e.g., ocular, inhalation, parenteral, dermal, transdermal, buccal, rectal, vaginal, sublingual, perilingual, nasal, topical administration, or oral administration. Parenteral administration includes intravenous, intraperitoneal, subcutaneous, and intramuscular administration.

In some instances, the pharmaceutical composition and additional therapeutic agents are administered at least one hour, two hours, four hours, six hours, 10 hours, 12 hours, 18 hours, 24 hours, three days, seven days, or fourteen days apart. The dosage and frequency of administration of each component can be controlled independently. For example, one compound or combination may be administered three times per day, while the second compound or combination may be administered once per day. Combination therapy may be given in on-and-off cycles that include rest periods so that the patient's body has a chance to recover from any as yet unforeseen side effects. The compounds may also be formulated together such that one administration delivers both the mucin glycoprotein-therapeutic agent composition and the additional therapeutic agent. Optionally, any of the agents of the combination may be administered in a low dosage or in a high dosage, each of which is defined herein.

The additional therapeutic agents described herein may be admixed with additional active or inert ingredients, e.g., in conventional pharmaceutically acceptable carriers. A pharmaceutical carrier can be any compatible, non-toxic substance suitable for the administration of the compositions of the present invention to a patient. Pharmaceutically acceptable carriers include, for example, water, saline, buffers and other compounds, described, for example, in the Merck Index, Merck & Co., Rahway, N.J. A slow release formulation or a slow release apparatus may be also be used for continuous administration.

The additional therapeutic regimen may involve other therapies, including modification to the lifestyle of the patient being treated.

### EXAMPLES

The following examples are provided for the purpose of illustrating the invention and are not meant to limit the invention in any way.

### Example 1

### Preparation of Mucin-ITF₁₅₋₇₃ for Ophthalmic Administration

The pharmaceutical composition of this example is prepared for ophthalmic administration (Table 1) and may be used in the form of eye drops.

TABLE 1

| Ingredient | Amount | |
|---|---|---|
| Mucin glycoprotein (MUC2) | 1-5% | (w/v) |
| ITF₁₅₋₇₃ | 20-50 | mg/ml |
| 0.1 N NaOH/0.1 N HCl | Variable | |
| NaCl | 286 (±30) | mOsm/kg |

| | |
|---|---|
| Sterile saline | Variable |

The eye drops are prepared by adding ITF₁₅₋₇₃ in a 0.9% sterile saline solution to a 1-5% solution of MUC2 in a 0.9% sterile saline solution, yielding a final ITF₁₅₋₇₃ concentration of 20-50 mg/ml. The solution is adjusted to a pH of 7.4 using 0.1 N sodium hydroxide (NaOH) and 0.1 N hydrochloric acid (HCl) and made isotonic with sodium chloride (NaCl). The solution is then filtered through a sterile filter (Millipore, USA; Catalog No. MPGL02 GH2). The filtered solution is dispensed into 8-ml amber Sano Dropper bottles in 1-ml aliquots for use as eye drops. The eye drops are administered topically for the treatment of, e.g., epithelial lesions on ophthalmic surfaces.

### Example 2

### Preparation of Mucin-ITF₁₅₋₇₃ Gel

The pharmaceutical composition of this example is prepared for topical administration to the oral mucosa (Table 2).

TABLE 2

| Ingredient | Amount | |
|---|---|---|
| Mucin glycoprotein (porcine) | 10% | (w/v) |
| ITF₁₅₋₇₃ | 10-50 | mg/ml |
| Glycerin | 10% | (v/v) |
| CaCl₂ | 1-10 | mM |
| 0.1 N NaOH/0.1 N HCl | Variable | |
| Benzylkonium chloride | 0.01% | (w/v) |

| | |
|---|---|
| Sterile water | Variable |

The gel is prepared by mixing a 10% porcine mucin solution with an ITF₁₅₋₇₃ solution, yielding a final ITF₁₅₋₇₃ concentration of 10-50 mg/ml. Both the porcine mucin solution and ITF₁₅₋₇₃ solution are prepared using sterile water. The mucin-ITF₁₅₋₇₃ solution is mixed using a Vortex mixer. Glycerin is slowly added to the mucin-ITF₁₅₋₇₃ solution as the solution is being mixed. Sufficient NaOH and HCl are added to the solution to adjust the pH to 6.5-7.5. The pH of the solution is measured with pH indicator paper. Benzylkonium chloride is added to 0.01% as a preservative. The solution equilibrates for 30 minutes at 20° C. After equilibration, the viscosity of the solution is measured in a viscometer.

The gel prepared in this example is used for topical administration to the oral mucosa and may also be used for topical administration to, e.g., the skin, vagina, cervix, and uterus, using an appropriate applicator.

### Example 3

### Preparation of Mucin-ITF₁₅₋₇₃ syrup

The pharmaceutical composition of this example is prepared for oral administration (Table 3).

TABLE 3

| Ingredient | Amount | |
|---|---|---|
| Mucin glycoprotein (MUC2) | 10% | (w/v) |
| ITF₁₅₋₇₃ | 20 | mg/ml |
| Citric acid monohydrate | 5 | g |
| Calcium gluconate | 10 | mM |
| Benzoic acid solution | 20 | ml |
| Lemon spirit | 1 | ml |
| Water | 20 | ml |

| | |
|---|---|
| Sorbitol solution | Variable |

The syrup is prepared by dissolving MUC2 and citric acid in water and slowly adding 250 ml of the sorbitol solution (70% (w/v) D-sorbitol in water). ITF₁₅₋₇₃ is dissolved in 500 ml of sorbitol. The benzoic acid is added to the ITF₁₅₋₇₃-sorbitol solution. The mucin and ITF₁₅₋₇₃ solutions are mixed and the citric acid monohydrate, calcium gluconate, and lemon spirit are added to the mixture. A sufficient volume of the sorbitol solution is added to yield a final volume of 1000 ml.

The syrup prepared in this example may be used for the treatment of, e.g., esophageal, gastric, and intestinal epithelial lesions.

### Example 4

### Preparation of Water-Soluble Mucin-ITF₁₅₋₇₃ Suppositories

The pharmaceutical composition of this example is prepared as a suppository (Table 4).

TABLE 4

| Ingredient | Amount | |
|---|---|---|
| Mucin glycoprotein (MUC1) | 20% | (w/v) |
| ITF₁₅₋₇₃ | 50 | mg/ml |
| Polyethylene glycol (PEG) 1000 | 9.75 | g |
| Polyethylene glycol (PEG) 4000 | 3.25 | g |

The mucin-ITF₁₅₋₇₃ mixture is prepared by adding 5 ml of a 100 mg/ml solution of ITF₁₅₋₇₃ in sterile water to 5 ml of a 40% solution of MUC1 in sterile water, yielding 10 ml of gel containing 20% (w/v) mucin and 50 mg/ml ITF₁₅₋₇₃. The PEG-1000 and PEG-4000 are melted by placing them into a water bath at 70° C. for 3 minutes. The melted PEGs are cooled to 45° C. and the mucin-ITF₁₅₋₇₃ solution is added. The resulting mixture is cast into a suppository mold and allowed to set at room temperature. The suppositories are stored at 4-10° C.

The suppositories may be used for, e.g., epithelial lesions in the lower gastrointestinal tract and female reproductive tract.

### Example 5

### Mucins as a Vehicle for Trefoil Peptide Delivery

We successfully performed the following procedure.

We gathered a study population composed of human volunteers to assess the adherence of ITF₁₅₋₇₃ to the mucus lining of the oral cavity. The ITF₁₅₋₇₃ preparation was formulated as a 40 mg/ml sterile aqueous solution with a pH between 6.0 and 6.5. The preparation was placed into a 3.5-ml buccal spray unit that dispensed 100 μl of the preparation per puff.

Enrolled subjects were instructed to administer three puffs of the buccal spray per administration with one puff to the inside of each cheek and one puff to the floor of the mouth. At various time intervals post-treatment (0, 2, 5, 10, 20, 30, 45, 60, 90, and 120 minutes), a firm swab of the oral mucosa on the cheek, floor of the mouth, and lateral tongue was made with a sterile nylon swab (Capon Innovation, Italy). The subjects were instructed to take a 100-ml drink of water prior to the 60-minute swab.

The swabs were resuspended in NuPage SDS Sample Buffer (Invitrogen, USA; Catalog No. NP0007) and fractionated using NuPage SDS-polyacrylamide gel electrophoresis (Invitrogen, USA; Catalog No. NP0302). The SDS-PAGE gel containing the fractionated buccal swab samples was transferred onto a nitrocellulose membrane using a Novex X-Cell II Mini-Cell Blot Module (Invitrogen, USA) at 30 Volts and 200 mAmps for 60 minutes. Tris-glycine, provided by the vendor, was used as the transfer buffer. The nitrocellulose membranes were blocked in 4% bovine serum albumin (BSA) in Tris-buffered saline containing 0.5% Tween-20. ITF₁₅₋₇₃ was detected on the nitrocellulose membrane by using a purified mouse monoclonal antibody probe that was raised against the human ITF₁₅₋₇₃ protein. ITF₁₅₋₇₃ immunoreactivity was detected by chemiluminescence using the SuperSignal West Pico reagent kit (Pierce, USA; Catalog No. 1856135) after application of the secondary antibody, Goat anti-mouse. The signal was scanned onto a ChemiDoc Gel Documentation System (Bio-Rad Laboratories, UK) for 1 to 10 minutes.

The results demonstrated that ITF₁₅₋₇₃ sprayed onto the buccal mucosa readily equilibrated into a mucin-bound compartment and a free dimeric form, based on densitometric scans of the respective lanes on the gel. The bulk of the ITF₁₅₋₇₃ sprayed onto the buccal mucosa was bound to mucins on the mucosal surface. The results suggest that there is an equilibrium between the bound and free pools of ITF₁₅₋₇₃, indicating that the mucin-bound ITF₁₅₋₇₃ serves as a reservoir or depot following buccal administration which continues to release free ITF₁₅₋₇₃ into the oral cavity over time. Therefore, the trefoil polypeptide-mucin glycoprotein compositions described herein are useful for delivering trefoil polypeptide to a mucosal surface and providing, e.g., an extended release mechanism that increases the therapeutic effect of the trefoil polypeptide.

### OTHER EMBODIMENTS

All publications, patents, and patent applications mentioned in the above specification are hereby incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

What is claimed is:

1. A method for treating a patient having a disorder of impaired mucin function, said method comprising administering to said patient a pharmaceutical composition comprising a mucin glycoprotein and a therapeutic agent, wherein said mucin glycoprotein and said therapeutic agent are present in amounts sufficient to treat said disorder.

2. The method of claim 1, wherein said disorder is a disorder of the stomach, intestines, distal bowel, mouth, or esophagus.

3. The method of claim 1, wherein said disorder is a disorder of the skin, vaginal epithelium, cervical epithelium, uterine epithelium, respiratory epithelium, or surface of the eye.

4. The method of claim 3, wherein said disorder is dry eye.

5. The method of claim 1, wherein said composition is administered to a site of impaired mucin function in said patient.

6. The method of claim 1, wherein said patient is human.

7. The method of claim 1, wherein said composition is formulated in unit dosage form.

8. The method of claim 1, wherein said composition is formulated for oral, buccal, topical, rectal, subcutaneous, vaginal, inhalation, ophthalmic, parenteral, intravenous, or intramuscular administration.

9. The method of claim 1, wherein said mucin glycoprotein is selected from the group consisting of MUC1, MUC2, MUC3A, MUC3B, MUC4, MUC5AC, MUC5B, MUC6, MUC7, MUC8, MUC9, MUC11, MUC12, MUC13, MUC15, MUC16, MUC17, MUC18, MUC19, and MUC20.

10. The method of claim 1, wherein said therapeutic agent is selected from the group consisting of a trefoil polypeptide, a chemotherapeutic agent, an anti-inflammatory agent, an antimicrobial agent, an antiviral agent, an antifungal agent, an analgesic, an anesthetic, a sedative, a lubricant, an immunomodulatory agent, a 5-aminosalicylate derivative, and a peptide.

11. The method of claim 1, wherein said therapeutic agent is a trefoil polypeptide.

12. The method of claim 11, wherein said trefoil polypeptide is selected from the group consisting of the intestinal trefoil factor (ITF) set forth in SEQ ID NO.: 1, the pS2 set forth in SEQ ID NO.: 2, the spasmolytic peptide (SP) set forth in SEQ ID NO.: 3, and biologically active fragments thereof.

13. The method of claim 12, wherein said trefoil polypeptide is selected from the group consisting of the ITF set forth in SEQ ID NO.: 1, and biologically active fragments thereof.

14. The method of claim 13, wherein said polypeptide is ITF₁₅₋₇₃.

15. The method of claim 1, further comprising administering an additional therapeutic agent to said patient concurrently or within fourteen days of administering said composition.

16. The method of claim 15, wherein said additional therapeutic agent is selected from the group consisting of a trefoil polypeptide, a chemotherapeutic agent, an anti-inflammatory agent, an antimicrobial agent, an antiviral agent, an antifungal agent, an analgesic, an anesthetic, a sedative, a lubricant, an immunomodulatory agent, a 5-aminosalicylate derivative, and a peptide.

17. A method of treating pain caused by or associated with a disorder of impaired mucin function, said method comprising administering to said patient a pharmaceutical composition comprising a mucin glycoprotein and a therapeutic agent, wherein said mucin glycoprotein and said therapeutic agent are present in amounts sufficient to treat said disorder.

18. A pharmaceutical composition comprising:
(i) a mucin glycoprotein; and
(ii) a therapeutic agent,
wherein said composition is formulated for administration to a patient.

19. The composition of claim 18, wherein said mucin glycoprotein and said therapeutic agent are present in amounts that, when administered to a patient, are sufficient to treat an epithelial lesion in said patient.

20. The composition of claim 18, wherein said mucin glycoprotein and said therapeutic agent are present in amounts that, when administered to a patient, are sufficient to treat a disorder of impaired mucin function in said patient.

21. The composition of claim 18, wherein said composition is formulated in unit dosage form.

22. The composition of claim 18, wherein said composition is sterile.

23. The composition of claim 18, wherein said composition is formulated as a pill, a powder, a granulate, a suspension, an emulsion, a solution, a gel, a paste, an ointment, a cream, a foam, a lotion, a plaster, a suppository, an enema, an injectable, an implant, a spray, or an aerosol.

24. The composition of claim 18, wherein said composition is formulated for oral, buccal, topical, rectal, subcutaneous, vaginal, inhalation, ophthalmic, parenteral, intravenous, or intramuscular administration.

25. The composition of claim 18, wherein said mucin glycoprotein is selected from the group consisting of MUC1, MUC2, MUC3A, MUC3B, MUC4, MUC5AC, MUC5B, MUC6, MUC7, MUC8, MUC9, MUC11, MUC12, MUC13, MUC15, MUC16, MUC17, MUC18, MUC19, and MUC20.

26. The composition of claim 18, wherein said therapeutic agent is selected from the group consisting of a trefoil polypeptide, a chemotherapeutic agent, an anti-inflammatory agent, an antimicrobial agent, an antiviral agent, an antifungal agent, an analgesic, an anesthetic, a sedative, a lubricant, an immunomodulatory agent, a 5-aminosalicylate derivative, and a peptide.

27. The composition of claim 18, wherein said therapeutic agent is a trefoil polypeptide.

28. The composition of claim 27, wherein said trefoil polypeptide is selected from the group consisting of the intestinal trefoil factor (ITF) set forth in SEQ ID NO.: 1, the pS2 set forth in SEQ ID NO.: 2, the spasmolytic peptide (SP) set forth in SEQ ID NO.: 3, and biologically active fragments thereof.

29. The composition of claim 28, wherein said trefoil polypeptide is selected from the group consisting of the ITF set forth in SEQ ID NO.: 1, and biologically active fragments thereof.

30. The composition of claim 29, wherein said polypeptide is ITF₁₅₋₇₃.

31. The composition of claim 18, wherein said therapeutic agent is not suitable for ophthalmic administration.


SEQUENCE LISTING (3 sequences)

SEQ ID NO: 1 - protein, 73 aa, Homo sapiens
MLGLVLALLSSSSAEEYVGLSANQCAVPAKDRVDCGYPHVTPKECNNRGCCFDSRIPGVPWCFKPLQEAECTF

SEQ ID NO: 2 - protein, 84 aa, Homo sapiens
MATMENKVICALVLVSMLALGTLAEAQTETCTVAPRERQNCGFPGVTPSQCANKGCCFDDTVRGVPWCFYPNTIDVPPEEECEF

SEQ ID NO: 3 - protein, 106 aa, Homo sapiens [sequence omitted]